(19) United States
(12) Patent Application Publication
(10) Pub. No.: US 2021/0244602 A1
(43) Pub. Date: Aug. 12, 2021

(54) Wearable Device for Inducing Eye Blinking

(71) Applicant: Justin Shing Chi CHENG, Hong Kong (CN)

(72) Inventor: Justin Shing Chi CHENG, Hong Kong (CN)

(21) Appl. No.: 16/788,295

(22) Filed: Feb. 11, 2020

(51) Int. Cl. A61H 5/00; A61B 5/00; A61N 1/04; A61N 1/36; A61H 23/02

(52) U.S. Cl. CPC A61H 5/00; A61B 5/0082; A61N 1/0452; A61N 1/0472; A61H 2201/165; A61H 23/02; A61B 2562/029; A61H 2230/605; A61H 2201/5092; A61N 1/36025

(57) ABSTRACT

Example embodiment provides a device for inducing blinking of a user. The device includes a sensor, a controller and a stimulator. The sensor is for measuring an ambient humidity. The controller is connected with the sensor for determining a blinking frequency for the user according to the ambient humidity and generating a control signal based on the blinking frequency. The stimulator is for providing a stimulus to the user according to the control signal. The stimulus induces blinking of the user.

### FIELD OF INVENTION

This invention relates to an electronic device, and in particular a wearable device for inducing eye blinking.

### BACKGROUND OF INVENTION

Neurotrophic keratopathy (NK) is corneal disorder characterized by a reduction or absence of corneal sensitivity, especially to pain and temperature. It is caused by the denervation of the sensory pathway of the corneal blinking reflex as a result of surgery, trauma, tumor invasion or infection. The motor pathway of the blinking reflex remains intact. The lack of sensory input or corneal hypoesthesia results in a reduction or loss of blinking reflex as the person does not feel the need to blink. This, in turn, will result in prolonged corneal exposure, dryness and a condition called exposure keratopathy which include serious complications like poor healing of corneal epithelial defects, corneal ulcers, infections and eventually permanent loss of vision as the protective mechanism of blinking response is lost.

Current treatment options include the use of frequent artificial tears which is cumbersome and ineffective. Surgical options including lateral tarsorrhaphy or upper lid metal weights implants are used to reduce the total area of corneal exposure. However, these are surgical operations which carries their own risks and can also cause cosmetic blemishes.

In view of the above, it would be highly desirable to develop a simple device that can induce natural blinking and solve the underlying problem.

### SUMMARY OF INVENTION

Devices and methods that can induce and regulate blinking are provided for the user to reduce or prevent the problem of corneal exposure keratopathy and dryness.

One example embodiment provides a device for inducing blinking of a user. The device includes a sensor, a controller and a stimulator. The sensor is for measuring an ambient humidity. The controller is connected to the sensor for determining a blinking frequency for the user according to the ambient humidity and generating a control signal based on the blinking frequency. The stimulator is for providing a stimulus to the user according to the control signal. The stimulus induces blinking of the user.

Another example embodiment provides a device for regulating blinking of a user. The device includes a first sensor, a second sensor, a controller and a stimulator. The first sensor is for measuring an ambient humidity. The second sensor is for detecting the blinking frequency of the user. The controller is connected to the first sensor and the second sensor for determining an optimal blinking frequency based on the ambient humidity, and generating a control signal when the blinking frequency of the user is lower than the optimal frequency blinking frequency. The stimulator is connected to the controller for providing a stimulus to the user to induce blinking of the user according to the control signal, so that the blinking frequency of the user is regulated to be not lower than the optimal blinking frequency.

Another example embodiment provides a method for inducing blinking of a user. The method includes the steps of: measuring an ambient humidity; determining a blinking frequency for the user according to the ambient humidity; generating a control signal based on the blinking frequency; providing a stimulus to the user according to the control signal; and inducing one blinking of the user by each stimulus.

### BRIEF DESCRIPTION OF FIGURES

FIG. 1 shows a block diagram of a device for inducing blinking of a user in accordance with an example embodiment.

FIG. 2 shows a block diagram of a device for regulating blinking of a user in accordance with an example embodiment.

FIG. 3A shows a front view of a pair of spectacles for inducing blinking of a user in accordance with an example embodiment.

FIG. 3B shows a top view of the spectacles of FIG. 3A.

FIGS. 4A and 4B show settings of the device for inducing blinking in accordance with an example embodiment.

FIG. 5 shows a setting of the device for inducing blinking in accordance with an example embodiment.

FIG. 6 shows a method for inducing blinking of a user in accordance with an example embodiment.

FIG. 7 shows a method for regulating blinking of a user in accordance with an example embodiment.

### DETAILED DESCRIPTION OF THE PREFERRED EMBODIMENTS

Example embodiments relate to devices that can induce the eye blinking of user, so as to reduce or prevent corneal exposure keratopathy and dryness.

As used herein and in the claims, “comprising” means including the following elements but not excluding others.

As used herein and in the claims, “connect” refers to electrical coupling or connection either directly or indirectly via one or more electrical means unless otherwise stated.

As used herein, “time of flight” refers to the measurement of the time taken by the infrared light to travel a distance through a medium.

Conventional treatments for NK or corneal hypoesthesia includes frequent use of artificial tears to keep the corneal and ocular surface lubricated and moist. Other treatment to reduce complications of corneal exposure includes implanting metal weight surgically to the upper eyelid to induce partial ptosis (droopy eyelid) or tarsorrhaphy. Both surgical options can reduce the corneal surface area exposed to air and hence, the area of cornea getting dry. However, the treatments can only be done surgically which carries its own risks and can also cause cosmetic blemishes.

Example embodiments solve the problem by providing a device that can induce natural blinking for the users that have the disease of NK or corneal hypoesthesia. The device includes a sensor, a controller and a stimulator. The sensor measures ambient humidity. Different ambient humidity requires different blinking frequency. The controller received information from the sensor, determines the blinking frequency required and generates a corresponding control signal. The stimulator provides stimulus to the user according to the control signal. The stimulus can induce blinking of the user. The device is wearable by the user, such as a pair of spectacles, and can provide a natural way for preventing exposure keratopathy caused by less/no blinking, while not burdening the user with frequent eye drops or change the appearance of the patient with surgical treatment options.

In one example embodiment, the stimulus is a conditioned stimulus associated with the blinking of the user by cognitive training.

In one example embodiment, the device further includes a sensor for detecting the blinking of the user and providing the information to the controller. Then the controller can adjust the control signal according to the information. By way of example, the sensor includes an emitter for emitting infrared light to the user's eyes, specifically, corneal or lid surface, and a receiver for receiving the infrared light reflected from those surfaces. The time of flight is registered by the sensor and is converted into an electronic signal, and the electronic signal is sent to the controller for determining if there is a blinking of the user.

In one example embodiment, the stimulator includes at least one electrode for providing the stimulus to the user's orbicularis oculi muscle, wherein the stimulus includes an electronic stimulating signal. The electronic stimulating signal is either a conditioned stimulus that is associated with blinking by cognitive training or an unconditional stimulus to stimulate the contraction of the orbicularis oculi muscle which incurs blinking. In another example embodiment, the stimulator includes at least one haptic motor for providing haptic stimulus to the user, wherein the haptic stimulus is a conditioned stimulus associated with the blinking of the user by cognitive training.

Example embodiment further provides a device for regulating blinking of a user. The device includes a first sensor, a second sensor, a controller and a stimulator. The first sensor is for measuring an ambient humidity. The second sensor is for detecting the blinking frequency of the user. The controller is connected with the first sensor and the second sensor for determining an optimal blinking frequency based on the ambient humidity, and generating a control signal when the blinking frequency of the user is lower than the optimal frequency blinking frequency. The stimulator is connected with the controller for providing a stimulus to the user to induce blinking of the user according to the control signal, so that the blinking frequency of the user is regulated to be not lower than the optimal blinking frequency.

In one example embodiment, the controller adjusts the control signal when the user fails to blink in response to the stimulus and the stimulator increases strength of the stimulus accordingly. In another example embodiment, the controller generates a control signal when no blinking is detected in a pre-determined period and the stimulator provides a stimulus to induce the blinking of the user accordingly. By providing blinking information of the user detected by the sensor to the controller as a feedback, the device can regulate the blinking of the user to a desired rate.

Example embodiment further includes a method for inducing blinking of a user. The method includes the following steps: measuring an ambient humidity; determining a blinking frequency for the user according to the ambient humidity; generating a control signal based on the blinking frequency; providing a stimulus to the user according to the control signal and inducing one blinking of the user by the stimulus.

Example embodiments induce blinking when necessary and keep the blinking frequency at an optimal level, so that the corneal exposure of the user can be prevented or minimized.

In the following, several example embodiments are described according to the figures. The same numbering in different figures is used to indicate the same part.

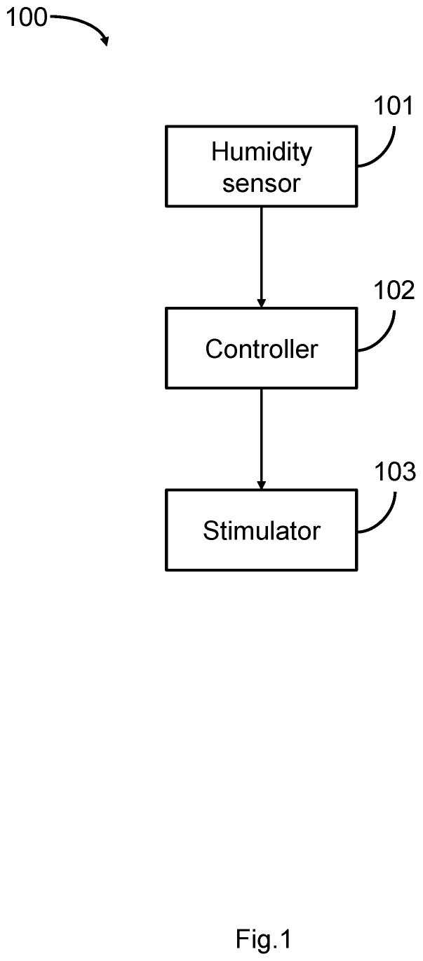

FIG. 1 shows a block diagram of a device 100 for inducing blinking of a user. The device 100 includes a humidity sensor 101, a controller 102 and a stimulator 103. The humidity sensor 101 is used for measuring the humidity of the environment. The controller 102 is connected with the humidity sensor 101 for determining an optimal blinking frequency for the user according to the ambient humidity and generating a control signal based on the blinking frequency. The stimulator 103 is connected with the controller 102, and provides a stimulus to the user according to the control signal. The stimulus is used to induce blinking of the user.

The ambient humidity is the factor for the controller 102 to decide how frequent the user have to blink. For example, if the humidity is relatively high, the user may blink less frequently as the air is moist and the eyes do not get dry so frequently. In comparison, if the humidity is relatively low, the eyes will get dry quickly, as a result, the user has to blink more frequently in order to keep the eye moistened.

The relationship between the humidity and the optimal blinking frequency can be pre-determined and stored in the controller 102 during the initial setup of the device 100. By way of example, the optimal blinking frequency is determined by assessing a tear breakup time (TBUT) of the user. The TBUT is recorded as the number of seconds that elapse between the last blink and the appearance of the first dry spot in the tear film over the cornea surface, which is the maximum time period that a person can tolerate before the next blink.

When the controller 102 determines the blinking frequency, it sends a control signal to the stimulator 103 accordingly. The stimulator 103 then generates a stimulus to the user, which induces blinking of the user.

In one example embodiment, the stimulator generates a conditioned stimulus that is associated with the blinking of the user by cognitive training. By way of example, cognitive training is used to achieve a conditioned reflex. The working principle is as follows. In patients with NK or corneal hypoesthesia, the sensory/afferent pathway of the corneal blinking reflex is damaged but the motor/efferent pathway is still intact. The “startle reflex” also remains intact in these patients. Therefore, one can still blink involuntarily when an object is being thrown at the patient face. The conditioned reflex starts with the first phase when the object is being thrown at the subject sitting in front of a transparent glass. The object is stopped by the glass, but the patient blinks, due to startled reflex. The second phase involves the application of a conditioning stimulus. The stimulus, such as a small current of electricity or a haptic vibration, is active only when the object is being thrown at the patient's face, by which blinking is induced. The third phase is the measurement of the conditioned reflex, in which the patient should blink with the stimulus in the absence of the object thrown at him.

By way of example, the stimulator 103 includes at least one electrode that provides electrical stimulus to stimulate the user's orbicularis oculi muscle. The electrical stimulus can be used to cause contraction of the orbicularis oculi muscle, so as to induce blinking of the user. Nevertheless, this way will consume too much electricity on the electrode with respect to the battery in a wearable device. Therefore, one example embodiment adopts the concept of cognitive training described above, and in the second stage, a small current of electricity is released from the electrode along with the object to create conditioning stimulus. In the last stage, the object will no longer go towards the user and only the small current of electricity is applied to the user from the electrode. Eventually, the user blinks in response to electricity. In this way, only little amount of electricity is needed to provide stimulus so that the battery life of the device can be substantially prolonged.

By way of example, the stimulator 103 includes at least one haptic motor for providing haptic stimulus to the user. The haptic stimulus is a conditioned stimulus associated with the blinking of the user by the cognitive training described above. Comparing with electrodes, the haptic motor is safer for the user as there is no current going through the subcutaneous skin tissue. The haptic motor can be controlled with drivers such as DRV2605L from Texas Instruments.

By way of example, the stimulator 103 may include other components or generate more than one kind of stimulus.

In one example embodiment, the device 100 further includes a sensor for detecting the blinking action of the user. By way of example, the sensor includes an emitter for transmitting infrared light to the user's ocular surface, and a receiver for receiving the infrared light reflected from the user's ocular surface. The time of flight is converted into an electronic signal and sent to the controller for determining if there is a blinking of the user. The working principle of the sensor is as follows: in the case of open eyes, the infrared light transmitted by the emitter is reflected by the cornea, while in the case of closed eyes, the infrared light is reflected by the eyelid, i.e., the reflected distance is shortened when the user blinks. The time of flight required for the infrared to be detected by the receiver will be shorter in a closed eye. Therefore, each change of the reflected distance, i.e., time of flight, can be counted as one blinking event. For example, the sensor is VL6180x from STMicroelectronics can be used as an infrared proximity sensor.

In one example embodiment, the controller 102 monitors the blinking of the user by the blinking detection sensor and determines if the blinking corresponds to the stimulus. When the user fails to blink in response to the stimulus is detected by the controller 102, the controller 102 adjusts the control signal to generate a stimulus with increased strength in the stimulator 103, such as a larger simulating current in the electrodes or a stronger vibration of the haptic motor, so that the user can sense the stimulus and blink accordingly.

In one example embodiment, the controller 102 monitors the interval between two subsequent blinkings of the user by the blinking detection sensor and generates a control signal when no blinking is detected in a pre-determined period and the stimulator provides a stimulus to induce the blinking of the user accordingly. By way of example, for an average relative humidity of 80%, a blink rate is 8-21 blinks per minute is considered as normal. A blink rate of less than 5 blinks per minute or 1 blink per 12 seconds is considered as the lower limit of normal. A blinking interval of 12 seconds triggers the controller 102 to send a control signal to the stimulator 103, and the stimulator 103 generates a stimulus to the user. As the tear evaporation decreases with the increase of environmental humidity, the optimal blinking frequency lowers with the increase of environmental humidity. Therefore, the blinking frequency that is determined by the controller 102 has a negative correlation with the ambient humidity that is measured by the humidity sensor 101. It can also be adjusted according to the user's TBUT on the initial setup of the device 100.

In one example embodiment, the stimulus is one pulse that induces one blinking, in other example embodiments, the stimulus may be a sequence of pulses that induces one blinking.

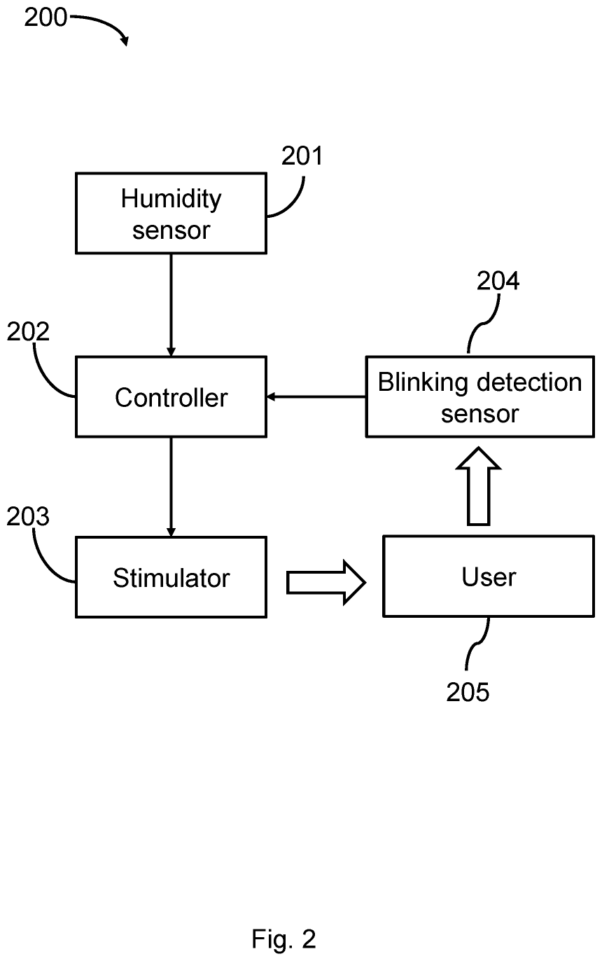

FIG. 2 shows a block diagram of a device 200 for regulating blinking of a user in accordance with an example embodiment.

The device 200 includes a humidity sensor 201, a blinking detection sensor 204, a controller 202 and a stimulator 203. The humidity sensor 201 is for measuring an ambient humidity. The blinking detection sensor 204 is for detecting an actual blinking frequency of the user. The controller 202 is connected with the humidity sensor 201 and the blinking detection sensor 204. It determines an optimal blinking frequency based on the ambient humidity, and compares the actual blinking frequency of the user with the optimal frequency. When the value of the former is lower than the latter, the controller 202 generates a control signal to the stimulator 203. The stimulator 203 then provides a stimulus to the user according to the control signal to induce blinking of the user.

By way of example, the humidity sensor 201 measures an environmental humidity of 75%. The controller 202 determines the optimal blinking frequency to be 10 blinks per minute at this humidity. When the blinking detection sensor 204 detects that the user does not blink over 6 seconds, the controller 202 will send a control signal to the stimulator 203 for generating a stimulus to the user. As the stimulus induces a blinking, the user can keep a blinking frequency not lower than the optimal blinking frequency eventually.

By way of example, the blinking detection sensor 204 is an infrared proximity sensor. The distance between the corneal surfaces to the sensor 204 is 12.5 mm, which is the back vertex distance (the distance between the back surface of the spectacles and the front of the cornea) used for making spectacle frames. Thickness of the upper lid is about 4-5 mm Therefore, when the eyelid is closed, there is a 30-40% reduction in the distance between the corneal surface to the sensor 204. Such a percentage of reduction is sufficient to identify eye blinking and is consistent even when the spectacle is not placed at exactly 12.5 mm from the cornea. Hence, the percentage of reduction rather than the actual distance is used for detection of blinking. For example, the distance information from the sensor 204 is continuously fed to the controller 202 for analysis. When there is a sudden reduction of distance or time of flight over 30%, a blink is counted.

By way of example, the humidity sensor continuously measures the humidity of the surrounding environment. When the humidity is lowered by 10%, the optimal blinking frequency is increased by 20% in order to compensate for the evaporative loss.

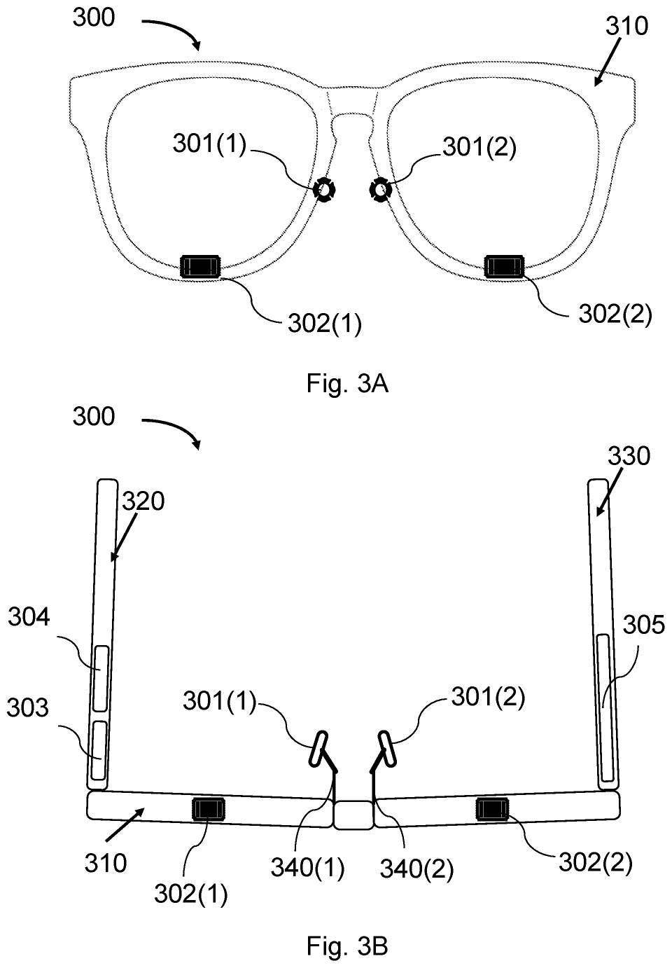

FIGS. 3A and 3B show a front view and a top view of a pair of spectacles 300 for inducing blinking of a user in accordance with an example embodiment.

As shown in FIGS. 3A and 3B, the spectacles 300 includes two haptic motors 301(1) and 301(2), two infrared sensors 302(1) and 302(2), a humidity sensor 303, a processor 304 and a battery 305. The haptic motors 301(1) and 301(2) mounted on the nasal pads for providing haptic stimulus to the user. The infrared sensors 302(1) and 302(2) mounted at the lower rim of the frame 310 for detecting the blinking action of the user. The humidity sensor 303 and the processor 304 are mounted on the right arm 320 of the spectacles 300. The battery 305 is mounted on the left arm 330 of the spectacles for providing power supply for the haptic motors 301(1) and 301(2), the infrared sensors 302(1) and 302(2), the humidity sensor 303, and the processor 304. For example, the battery 305 is a rechargeable lithium battery.

The haptic motors 301(1) and 301(2) are connected to the processor 304 via a haptic motor driver. The haptic motors 301(1) and 301(2) are physically attached to the nasal pads of the spectacles 300 which, in turn are attached to the nasal bridge of the spectacle 300 via soft silicon pieces 340 (1) and (2). The soft silicon pieces 340 (1) and (2) reduced the vibration transmitted to the spectacle frame 310, so the vibration is concentrated on the nasal pad. The haptic motors 301(1) and 301(2) are rested on top of the nasal segment of the orbicularis oculi and apply a sensation of small, medium or strong buzz thereto.

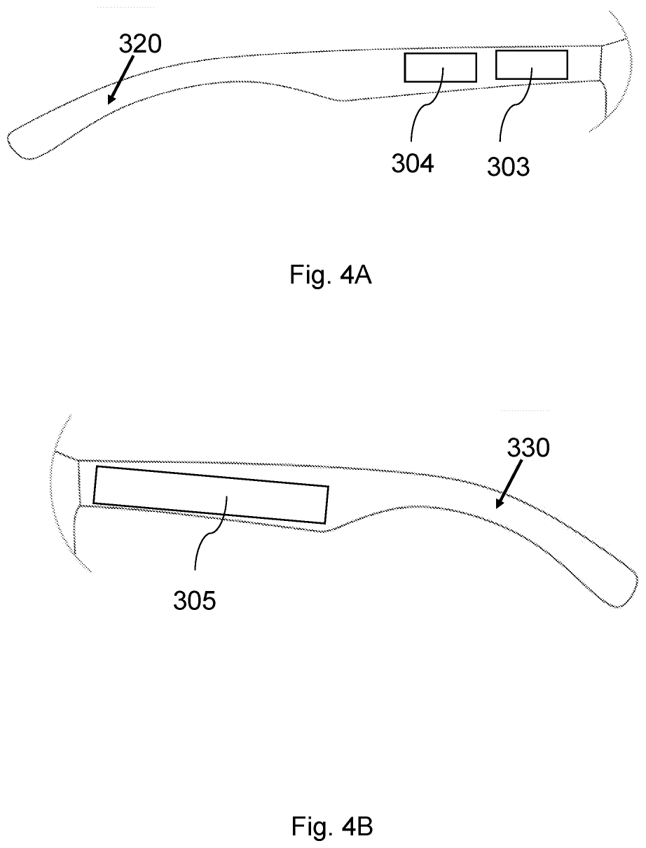

By way of example, the humidity sensor 303 and the processor 304 are mounted on the outer surface of the right arm 320 as shown in FIG. 4A. The battery 305 is mounted on the outer surface of the left arm 330 as shown in FIG. 4B. In other example embodiment, the haptic motors 301(1) and 301(2) may be configured on the left arm 320 or the right arm 330 of the spectacles, at the positions contacting the user's skin. The infrared sensors 302(1) and 302(2) may be configured at any other position on the frame 310 where the blinking of the user can be detected. The humidity sensor 303, the processor 304 and the battery 305 may not be the same as in FIGS. 3A and 3B. For example, the humidity sensor 303 or the processor 304 may not be attached to the spectacles 300 and may communicate with each other via wireless communication.

The infrared sensors 302(1) and 302(2) detects blinking action of the user and sends the information to the processor 304. The processor 304 receives the information and determines the interval between each two subsequent blinking. If the interval is longer than a pre-determined period, the processor 304 sends a control signal to the haptic motors 301(1) and 301(2) to generate a stimulus to the user. Consequently, blinking of the eyes is induced by the stimulus. By way of example, the predetermined period is 12 seconds. It can also be adjusted according to the user TBUT on the initial setup of the device. By way of example, the stimulus includes forces, vibrations, or motion generated by the haptic motors 301(1) and 301(2).

In one example embodiment, the spectacles 300 include only one infrared sensor since normally the two eyes of one user blink simultaneously instead of individually.

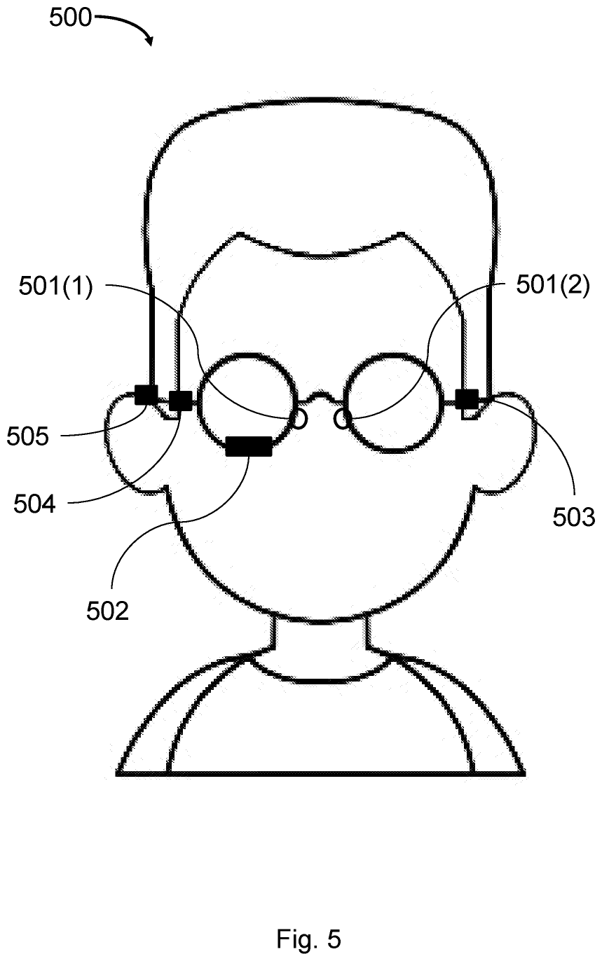

FIG. 5 shows a setting of the device 500 on a user for inducing or regulating blinking in accordance with an example embodiment.

In FIG. 5, the device 500 is a pair of spectacles which include two electrodes 501(1) and 501(2), one infrared sensor 502, a humidity sensor 502, a processor 504 and a battery 505. The two electrodes 501(1) and 501(2) are two metal nose plates which are structural parts of the spectacles that sit on the nasal part of the orbicularis oculi, so that the electrodes 501(1) and 501(2) are always in contact with the skin and the underlying muscle as the whole spectacles rest on it. The infrared sensor 502 is mounted at the lower rim of the frame of the spectacles for detecting the blinking action of the user. The humidity sensor 503, the processor 504 and the battery 505 are configured on the arms of the spectacles respectively.

In some example embodiment, the electrodes 501(1) and 501(2) are configured to be in contact with the skin/muscles around the eyes, such as orbicularis oculi, and provides electrical current to contract the muscle, so that an unconditioned blinking is incurred. In other example embodiment, the electrodes 501(1) and 501(2) provides a much smaller current to stimulate the skin nerves, and a conditioned blinking is incurred by a previous cognitive training of the user.

In one example embodiment, the number of the electrodes may be more or less than two and may be positioned on other parts of the user's body, as long as the stimulus it provides can be associated with the blinking of the user.

In one example embodiment, the infrared sensor may be mounted on the left side of the frame or right side of the spectacles frame only for detecting the blinking of the left eye or right eye only. In other example embodiments, the spectacles include two infrared sensors, which are mounted on each side of the spectacles frame respectively for detecting the blinking of both eyes.

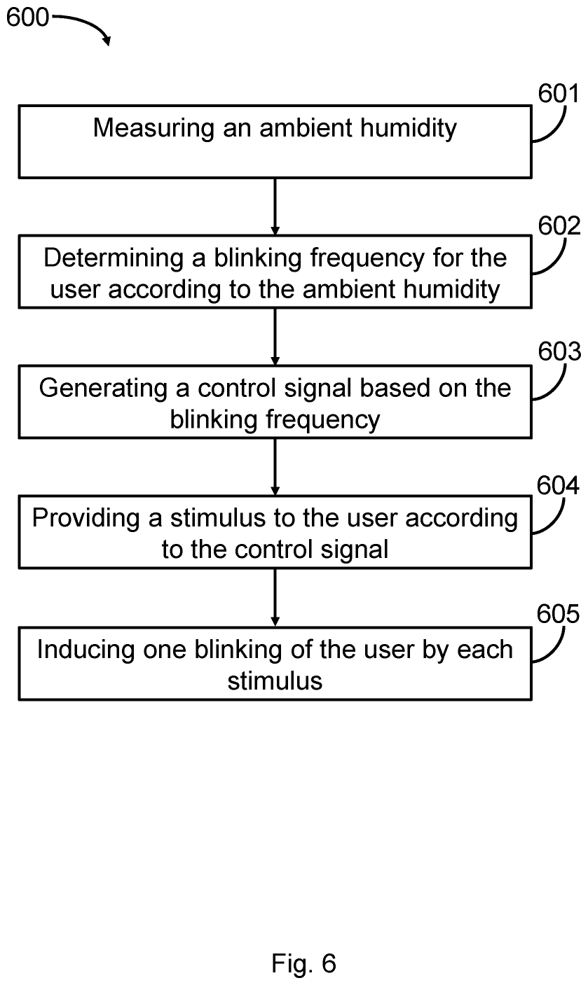

FIG. 6 shows a method for inducing blinking of a user in accordance with an example embodiment.

Block 601 states measuring an ambient humidity. For example, the ambient humidity is measured by a humidity sensor. The humidity sensor is configured nearby the user's eyes.

Block 602 states determining a blinking frequency for the user according to the ambient humidity. For example, the blinking frequency is determined by a controller or a processor and is an optimal blinking frequency under the ambient humidity.

Block 603 states generating a control signal based on the blinking frequency. By way of example, the controller generates a control signal.

Block 604 states providing a stimulus to the user according to the control signal. By way of example, a stimulator such as an electrode or haptic motor provides the stimulus. And the stimulus includes an electrical current, a force, a vibration, a motion or a buzz, etc. that can be sensed by the user.

Block 605 states inducing one blinking of the user by each stimulus. By way of example, the stimulus is a conditioned one that is associated with blinking of the user by cognitive training as described above. Each stimulus induces one blinking of the user so that the user can blink at the optimal frequency that is determined by the controller.

In one example embodiment, the blinking of the user is monitored by a sensor and fed back to the controller. If the blinking of the user is not in response to the stimulus, the controller will adjust the control signal to increase the intensity of the stimulus, so as to ensure the blinking of the user at the optimal frequency determined by the controller.

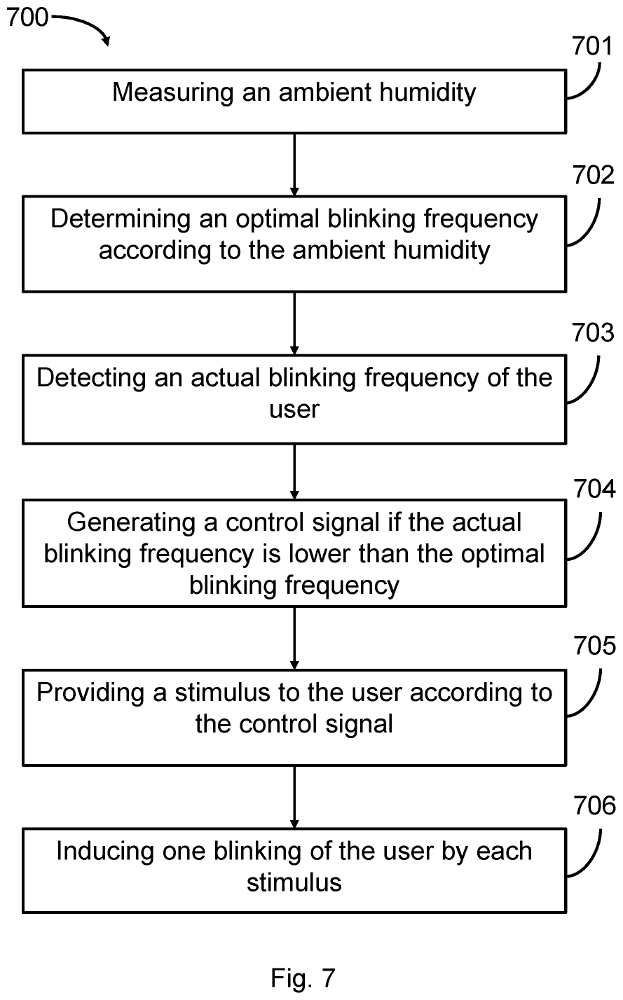

FIG. 7 shows a method for regulating blinking of a user in accordance with an example embodiment.

Block 701 states measuring an ambient humidity. For example, the ambient humidity is measured by a humidity sensor. The humidity sensor is configured nearby the user's eyes.

Block 702 states determining an optimal blinking frequency for the user according to the ambient humidity. For example, the optimal blinking frequency is determined by a controller or a processor and is negatively correlative with the ambient humidity.

Block 703 states detecting an actual blinking frequency of the user. By way of example, the blinking action of the user is detected by infrared sensors. The actual frequency of blinking is determined by a controller or a processor based on the sensors' measurements.

Block 704 states generating a control signal if the actual blinking frequency is lower than the optimal blinking frequency. By way of example, the controller or processor generates a control signal when the blinking interval of the user is longer than a pre-determined time period.

Block 705 states providing a stimulus to the user according to the control signal. By way of example, a simulator receives the control signal from the controller or processor and generates the stimulus.

Block 706 states inducing one blinking of the user by each stimulus. By way of example, the stimulus is a conditioned stimulus or an unconditioned one that can induce blinking of the user. By this way, the blinking frequency of the user is regulated to be not lower than the optimal value, so that the user's eyes are prevented from dryness caused by evaporation.

The exemplary embodiments of the present invention are thus fully described. Although the description referred to particular embodiments, it will be clear to one skilled in the art that the present invention may be practiced with a variation of these specific details. Hence this invention should not be construed as limited to the embodiments set forth herein.

1-17. (canceled)

18. A method for treating a human patient with Neurotrophic keratopathy (NK) or corneal hypoesthesia, comprising:
(a) stimulating a motor efferent pathway of a corneal blinking reflex of the patient to create a startle reflex by throwing an object to the patient's face in the patient such that a blinking of the eyes is induced;
(b) applying a stimulus to the patient simultaneously as the blinking occurs;
(c) measuring whether the patient blinks when the stimulus is applied without the object being thrown through conditioned reflex;
(d) determining a tear breakup time (TBUT) of the patient at various ambient humidity;
(e) providing a pair of spectacles for regulating blinking frequency, the spectacles comprising:
(i) a first sensor for measuring an ambient humidity;
(ii) a controller connected with the first sensor for determining the blinking frequency for the patient according to the ambient humidity and generating a control signal based on the blinking frequency; and
(iii) a stimulator for providing the stimulus to the patient according to the control signal, wherein the stimulus induces the blinking of the patient;
(f) inputting into the spectacles:
(i) the control signal according to result of the conditioned reflex in the patient;
(g) mounting the spectacles onto the patient;
(h) generating, by the controller, the control signal based on the ambient humidity and the TBUT of the patient;
(i) providing, by the stimulator, the stimulus to the patient according to the control signal; and
(j) inducing, by the stimulus, the blinking of the patient;
wherein a larger electrical stimulus is an electrical current that consumes a larger amount of electricity that is sufficient for stimulating the patient's orbicularis oculi muscle so that an unconditioned blinking is incurred;
wherein a smaller electrical stimulus is a smaller current to stimulate skin nerves and not cause unconditioned blinking, but is able to cause the patient to blink after the conditioned reflex so that a battery life of the spectacles can be prolonged; and
wherein the stimulus in all the steps above is the smaller electrical stimulus and the larger electrical stimulus is never applied.

19-21. (canceled)

22. The method according to claim 18, wherein the stimulator includes at least one haptic motor for providing haptic stimulus to the patient, wherein the haptic stimulus is the stimulus associated with the blinking of the patient by cognitive training.
